(19) United States
(12) Patent Application Publication
(10) Pub. No.: US 2015/0216207 A1
(43) Pub. Date: Aug. 6, 2015

(54) ENZYME GRANULE BLENDS CONSISTING ESSENTIALLY OF SODIUM SULFATE

(71) Applicant: Danisco US Inc., Palo Alto, CA (US)

(72) Inventor: Nathaniel T. Becker, Hillsborough, CA (US)

(73) Assignee: DANISCO US INC., Palo Alto, CA (US)

(21) Appl. No.: 14/616,286

(22) Filed: Feb. 6, 2015

Related U.S. Application Data
(63) Continuation of application No. 14/000,458, filed on Nov. 5, 2013, now abandoned.

(30) Foreign Application Priority Data
Mar. 10, 2011 (CN) 2011/071678

(51) Int. Cl. A23K 1/165; A23K 1/00; C12N 9/48; C12N 9/16; C11D 3/386; C11D 3/04

(52) U.S. Cl. CPC A23K 1/1653; C11D 3/38672; C11D 3/046; C12N 9/48; C12N 9/16; A23K 1/004; C12Y 301/03

(57) ABSTRACT

The present teachings provide ways of improving the distribution of high payload enzyme granules by reducing their size, and by mixing them with size-matched dummy particles containing sodium sulfate. The present teachings also provide methods of using the mixtures.

### CROSS-REFERENCE TO RELATED APPLICATIONS

The present application is a continuation of U.S. patent application Ser. No. 14/000,458, filed Nov. 5, 2013, which is a National Stage of International Application No. PCT/US2012/027073, filed on Feb. 29, 2012, which claims the benefit of International Patent Application No.: PCT/CN2011/071678, filed on Mar. 10, 2011, and which are incorporated by reference in their entirety.

### TECHNICAL FIELD

The present teachings relate to the field of enzyme granules, and improved compositions with reduced cost and improved functionality. Methods of use are also provided.

### BACKGROUND

There is a need for lower cost enzyme granules for use in a variety of applications, including detergents, textiles, baking and steam-pelleted animal feed. These applications generally benefit from enzymes that are protected from moisture, temperature, and harsh chemicals. Accordingly, the enzyme is generally granulated and coated with one or more protective coatings. Protection of workers from exposure to sensitizing enzyme dusts is also advanced by coating. However, granulation and coating add significant costs to enzyme products. One means of reducing the cost of coated enzyme granules is to produce granules with a high enzyme activity, such that the cost of granulation and coating (both process costs and raw material costs) are reduced relative to a given cost of active enzyme.

Granular enzymes are incorporated into powdered products such as detergents, textile and baking mixes, and animal feed mashes or pelleting mixtures, by means of batch mixing or continuous metering equipment. Batch mixers can include tumbling mixers, conical or V-blenders, ribbon mixers and the like. Continuous mixers can include vibratory feeders, screw conveyors and other loss-in-weight or volumetric dosing mixers. At low incorporation ratios, it becomes difficult to deliver a consistent concentration of enzyme active per unit dose. Increased variability in active enzyme concentration is a consequence of not only process control limitations, but also of the statistical likelihood of delivering a substantial number of individual granules within a sample volume that corresponds to a typical application dose of powdered product. For example, if a dose of powdered particle contained 1000 particles, and the enzyme was present at a low dose such as 0.5%, there would be an average of 5 enzyme granules per dose of product, but from dose to dose, some doses would contain more than 5 enzyme granules, and others less than 5 enzyme granules—perhaps as low as zero or 1 particle in some doses.

In the context of animal feed, the variability that can arise from low numbers of enzyme granules in a single given feed dose (a single “feeding”) can be quite extreme. For example, many systems for metering enzyme granules into products are designed for a limited incorporation, and can handle enzyme granules containing up to only about 1-2 percent w/w active enzyme. An example can be illustrative. Say a single dose of chicken feed is roughly 50 grams. And, enzyme granules have an incorporation ratio in chicken feed of about 0.005 percent (ie—0.50 grams of enzyme granule per metric ton of chicken feed). Given that 10,000 enzyme granules typically weigh approximately one gram, then a typical chicken feed dose of 0.005 percent of 50 grams, or 2.5 milligrams, will contain only about 25 enzyme granules. Oversampling or undersampling the number of enzyme granules in a given chicken feed dose by a mere five enzyme granules, therefore, represents 20 percent variability in either direction, which can be an undesirable and commercially relevant degree of variation. If, in order to reduce costs, it is desired to increase the payload of these feed enzyme granules by a factor of ten, from 1% w/w to 10% w/w, this would reduce the number of enzyme granule in a dose of chicken feed to only 2-3 granules per dose. Normal dosing variability at this level could result in some doses of chicken feed containing little or no enzyme, while other doses might contain double the target concentration. This illustrates the motivation for increasing the number of particles per dose, by reducing the particle size of enzyme granules in animal feed. Similar calculations for the dosing of enzyme granules in other applications such as detergents, textiles, and baking provide motivation for the use of smaller, high payload granules to increase the number and distribution of enzyme granules in those applications as well.

Because of the limited incorporation ratio of many metering systems, it can be desirable to dilute the enzyme granules with an inactive particle that lacks enzyme, sometimes called a “dummy particle” However, as the activity of enzyme granules increase, the number of enzyme granules needed to deliver a given concentration of enzyme to a product (e.g. a detergent product or animal feed product) correspondingly decreases. This reduction in the number of enzyme granules per volume of product exacerbates the distribution problem and can result in a commercially unacceptable distribution (e.g.-a high variability in concentration between samples of the “same” product). Diluting the high payload enzyme granules with dummy particles addresses the metering constraints of customers who incorporate enzymes in their products, but it does not address the distribution problem, since the number of enzyme granules per application dose in the end product depends only upon the actual amount of enzyme added to the detergent per volume of final product, and is not influenced at all by the addition of dummy particles that have been added as a metering diluent.

### SUMMARY

The present teachings provide a mixture consisting essentially of; a small enzyme granule, wherein at least 80% of the small enzyme granule comprises a diameter of about 300-400 microns; and, a size-matched sodium sulfate dummy particle, wherein at least 80% of the size-matched sodium sulfate dummy particle comprises a diameter of about 300-400 microns, wherein the median size of the small enzyme granule and the median size of the sodium sulfate dummy particle are size-matched such that they vary by less than 20 microns.

In some embodiments, the sodium sulfate is anhydrous.

In some embodiments, the small enzyme granule comprises a sodium sulfate core, and at least one layer surrounding the core, wherein the at least one layer surrounding the core comprises enzyme.

In some embodiments, the enzyme is a protease.

In some embodiments, the sodium sulfate is anhydrous, and the sodium sulfate core has at least one layer surrounding it, and the enzyme is a protease. In some embodiments, the sodium sulfate is anhydrous, and the enzyme is a protease. In some embodiments, the sodium sulfate has at least one layer surrounding it and the enzyme is a protease.

In some embodiments, the present teachings provide a method of washing dishes comprising contacting the dishes with the mixture according to the present teachings.

In some embodiments, the present teachings provide a method of washing clothes comprising contacting the clothes with the mixture according to the present teachings.

In some embodiments, the present teachings provide a method of feeding animals comprising providing an animal feed to an animal in need of such feed, wherein the feed comprises the mixture according to the present teachings.

### BRIEF DESCRIPTION OF THE FIGURES

FIG. 1 shows some illustrative data according to the present teachings.

FIG. 2 shows some illustrative data according to the present teachings.

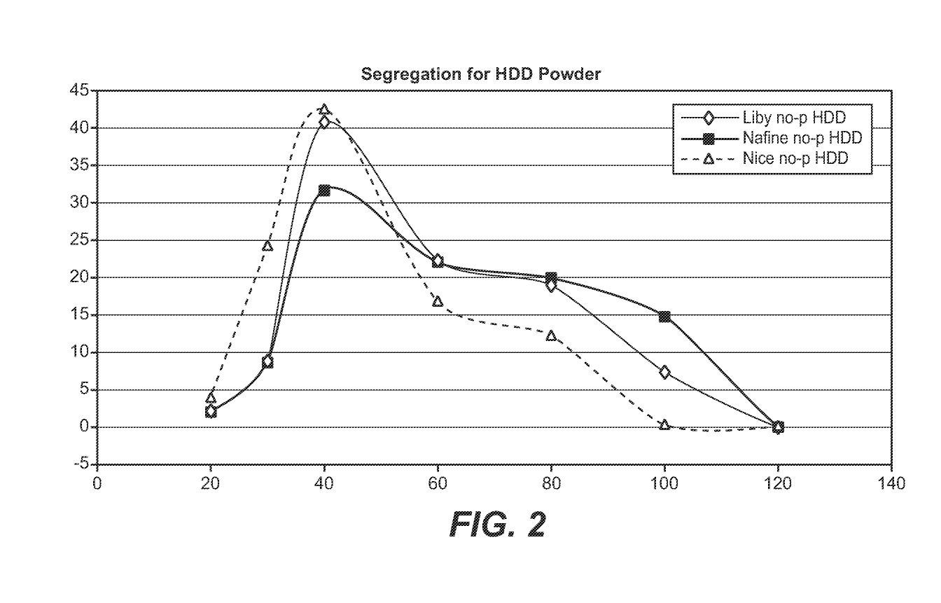

FIG. 3 shows some illustrative data according to the present teachings.

FIG. 4A shows some illustrative data according to the present teachings.

FIG. 4B shows some illustrative data according to the present teachings.

FIG. 5 shows some illustrative data according to the present teachings.

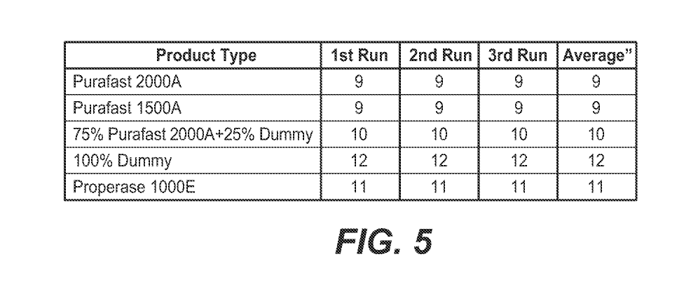

FIG. 6 shows some illustrative data according to the present teachings.

FIG. 7 shows some illustrative data according to the present teachings.

FIG. 8A shows some illustrative data according to the present teachings.

FIG. 8B shows some illustrative data according to the present teachings.

FIG. 8C shows some illustrative data according to the present teachings.

FIG. 9 shows an illustrative flow diagram according to the present teachings.

### DETAILED DESCRIPTION

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions

As used herein, the term “small enzyme granule” refers to a granule containing an enzyme with a median size (diameter) of around 200-450, 225-450, 250-450, 275-450, 300-450, 325-450, 350-450, 375-450, 400-450, 425-450, 200-225, 200-250, 200-275, 200-300, 200-325, 200-350, 200-375, 200-400, 200-425, 225-424, 250-400, 275-375, or 300-350. In some embodiments, the median size is less than 400 microns, for example 300-400 microns, and at most 20% are larger than 400 microns. In some embodiments, the median size is less than 400 microns, for example 300-400 microns, and at most 10% are larger than 400 microns.

As used herein, the term “size-matched” refers to the close similarity between the diameter size of the enzyme granule and the diameter size of the blending salt. In some embodiments, the median size of the enzyme granule and the median size of the blending salt are size-matched such that they vary by less than 40 microns. In some embodiments, the median size of the enzyme granule and the median size of the blending salt are size-matched such that they vary by less than 20 microns.

As used herein, “dummy particle” refers to an enzyme-lacking particle that is size-matched with an enzyme granule. One example of a blending salt is sodium sulfate, readily commercially available from Hanhua.

### Exemplary Embodiments

The present teachings provide one attractive way to improve the distribution of high payload enzyme granules by reducing their size, and by mixing them with size-matched dummy particles. The present teachings provide for several advantages. For example, producing several grades of enzyme granules at different enzyme payloads has historically required separate production and inventorying for each separate payload product. This is costly and laborious. By contrast, the present teachings provide that a single batch of high payload enzyme granules can be blended at different ratios with the size-matched dummy particles to produce “blend to order” products on a just-in-time basis, greatly streamlining production and inventory demands. The mixture of the present teachings provides minimum segregation, matched appearance, homogenous distribution, low cost, and operational simplicity. In addition, the particular salt(s) chosen for the blending particle provide for control of moisture so as to minimize activity loss of the enzyme due to moisture-mediated processes such as denaturation, aggregation, and chemical reaction with water soluble oxidants, surfactants, or other reactive species.

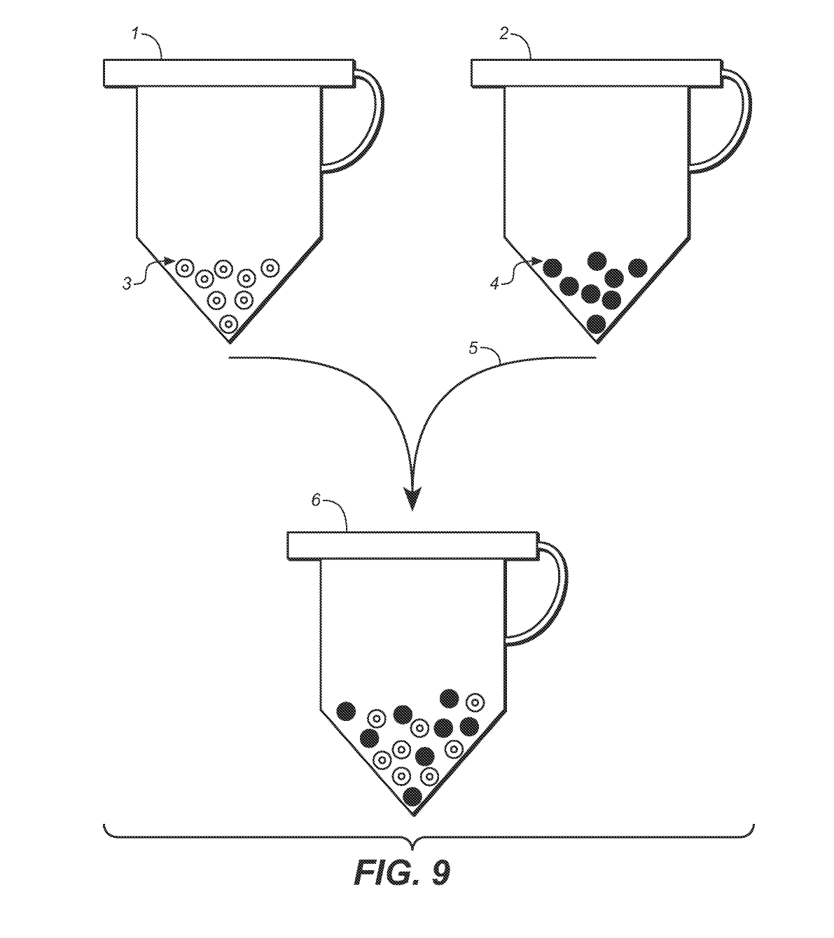

One embodiment according to the present teachings is depicted in FIG. 9. Here, a first source (1) containing small enzyme granules (nested circles, (3)), and a second source (2) containing dummy particles (solid circles, (4)) are blended together (5) to form a mixture (6)). The resulting mixture contains roughly equivalent numbers of particles, and the particles are roughly the same size. In various embodiments, the ratio will vary with the higher or lower batch mixing or continuous metering needs of the downstream end-user (eg—consumer detergent manufacturer).

In some embodiments, the present teachings provide a mixture comprising a small enzyme granule and a size-matched-dummy particle. In some embodiments the small enzyme granule is made according to WO2009/102770, which is hereby incorporated by reference in its entirety for any purpose. In some embodiments, the small enzyme granule is made with (a) a sodium sulfate salt crystal (alternately called a “seed” or “core”), (b) a coating layer or layers of enzyme(s), and (c) optional additional coatings, and the total added mass of (b) and (c) is less than 20% of the active enzyme particles. In some embodiments, the small enzyme granule is made via any of a variety of approaches for making enzyme granules, including for example those described in U.S. Pat. No. 5,324,649, which is hereby incorporated by reference in its entirety for any purpose.

In some embodiments, the present teachings provide a mixture consisting of, or consisting essentially of, a small enzyme granule and a size-matched-dummy particle, wherein the size-matched salt is sodium sulfate.

In some embodiments, the present teachings provide a mixture consisting of an enzyme granule made according to WO2009/102770, and a size-matched dummy particle, wherein the size-matched blending particle is sodium sulfate.

In some embodiments, the sodium sulfate is anhydrous. Anhydrous sodium sulfate can offer advantages in high humidity environments and provide for enzyme stability. Below about 75% RH, the anhydrous sodium sulfate won't absorb and retain significant amounts of water that could potentially reduce enzyme stability. Only at fairly high relative humidity, for example above 75% humidity, will the anhydrous sodium sulfate begin to absorb water, and even in such circumstances the high water binding capacity of this salt will provide a buffer or temporary sink for water which, while ultimately undesirable, nonetheless can to a certain extent and for some interval of time delay direct exposure of the enzyme to moisture-induced inactivation, thereby providing significant protection to the enzyme.

In some embodiments, the sodium sulfate in an anhydrous form, or a mixture of anhydrous and hydrated forms when blended with the enzyme granule. For example, the sodium sulfate will be substantially anhydrous when the humidity during storage is less than about 75% RH.

Any of a variety of enzymes can be included in the enzyme granules of the present teachings, including proteases, alpha amylases, aryl esterases, phytases, xylanases, cellulases, glucoamylases, pullulanases, beta amylases, and generally any enzyme of interest.

### EXAMPLES

### Example 1

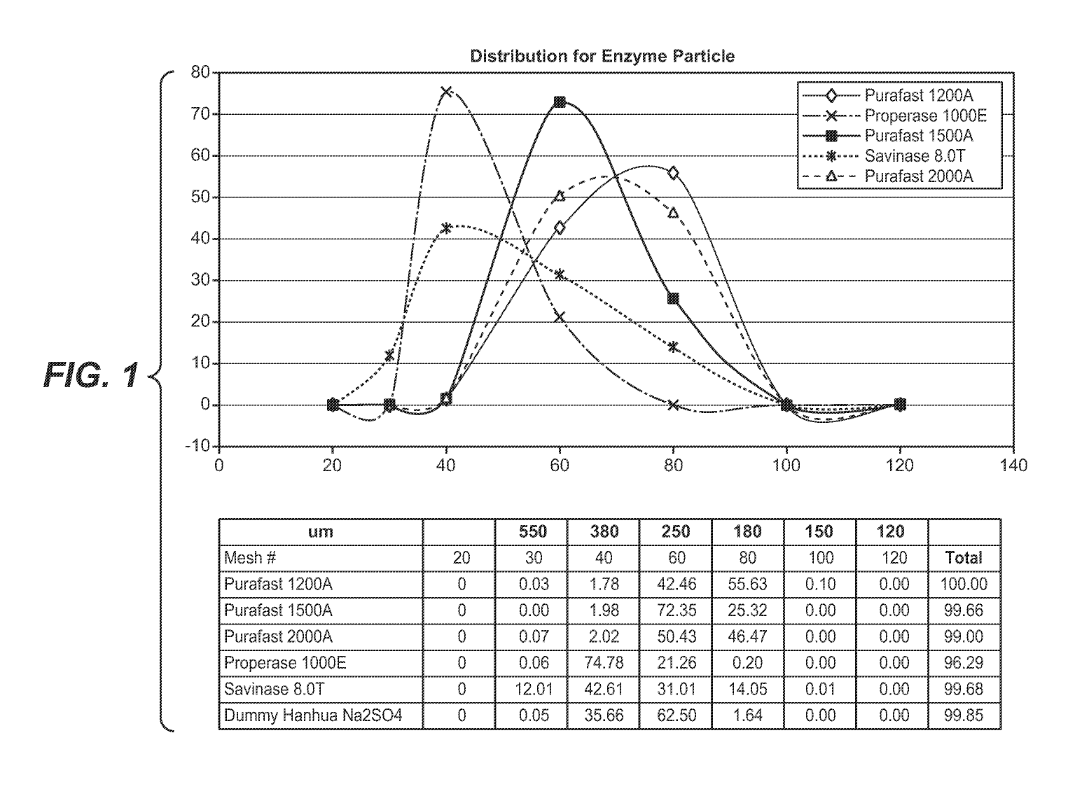

Size distribution of enzyme granules The particle size distributions of five different granular enzyme products were measured using sieve analysis, using U.S. standard sieve measurements. Mesh conversions to microns are shown in Table 1. The size distributions for three different spray-coated fluidized bed granules (Properase 1000E, Purafast 1200A, Purafast 2000A), one wet granulated matrix granule (Savinase 8.0T) and a blend of an enzyme granule with dummy particles (Purafast 1500A) are shown in FIG. 1. The Purafast 1500A blend was produced by blending 75% Purafast 2000A with 25% sodium sulfate dummy particles. The sodium sulfate dummy particles were a +40/−60 sieve cut of sodium sulfate crystals from Hanhua Corporation (China).

FIG. 1 shows that the mean particle size and size distribution of the Purafast 1500A blend is similar to that of the unblended pure enzyme granule product Purafast 2000A, and both have a significantly lower mean particle size than that of other enzyme products such as Purafect 1000E and Savinase 8.0T

### Example 2

### Size Distribution of Detergent Powders

The attached particle size diagram shows the particle size distribution of three standard Chinese heavy duty (HDD) laundry detergents, showing the mass percentage of particles on each U.S. standard mesh screen after sieving:

### Example 3

### Bulk Density of Enzyme Granules and Diluent Particles

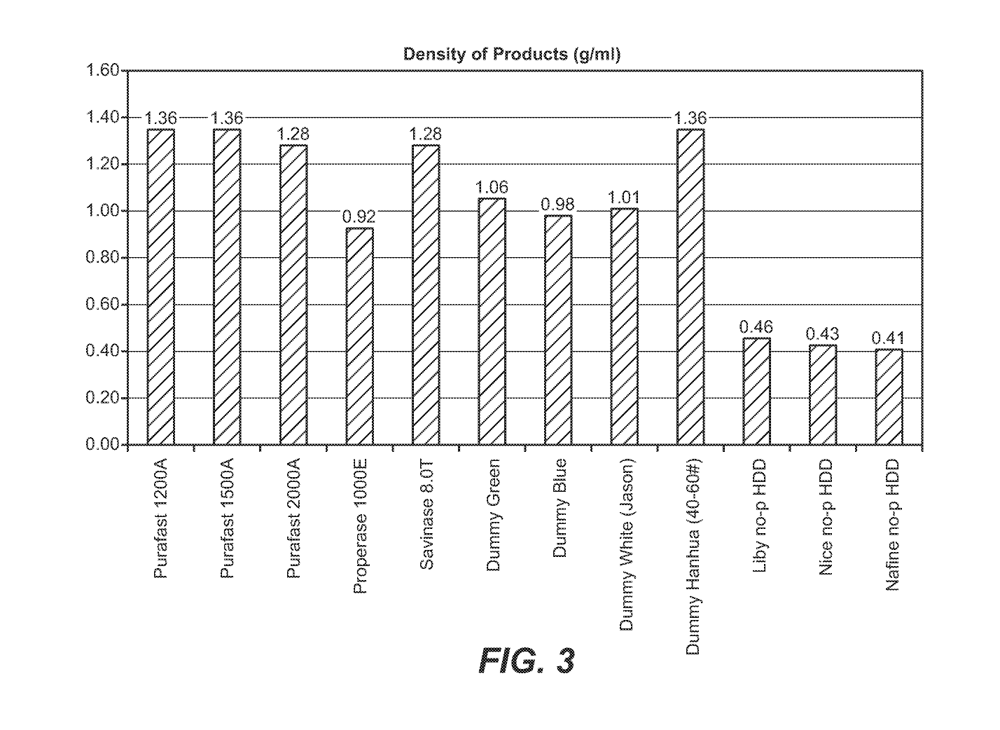

FIG. 3 shows a comparison of the bulk densities of several enzyme granules (Purafast 1200A, Purafast 2000A, Purafast 1000E, Savinase 8.0T), an enzyme granule blend (Purafast 1500A, defined in Example 1), dummy particles (green, blue and white placebo particles, and Hanhua +40/−60 mesh sodium sulfate crystals), and commercial laundry detergents (Liby no-phosphate HDD, Nice no-phosphate HDD and Nafine no-phosphate HDD). Bulk densities are tapped densities shown in units of grams per cubic centimeter. The figure demonstrates that the bulk densities of the Purafast 2000A and Hanhua −40/+60 mesh sodium sulfate are closely matched, as is the 75%/25% blend of these two, represented by the Purafast 1500A blend.

### Example 4

### Segregation Testing of Unblended and Blended Enzyme Particles in Detergent Powder

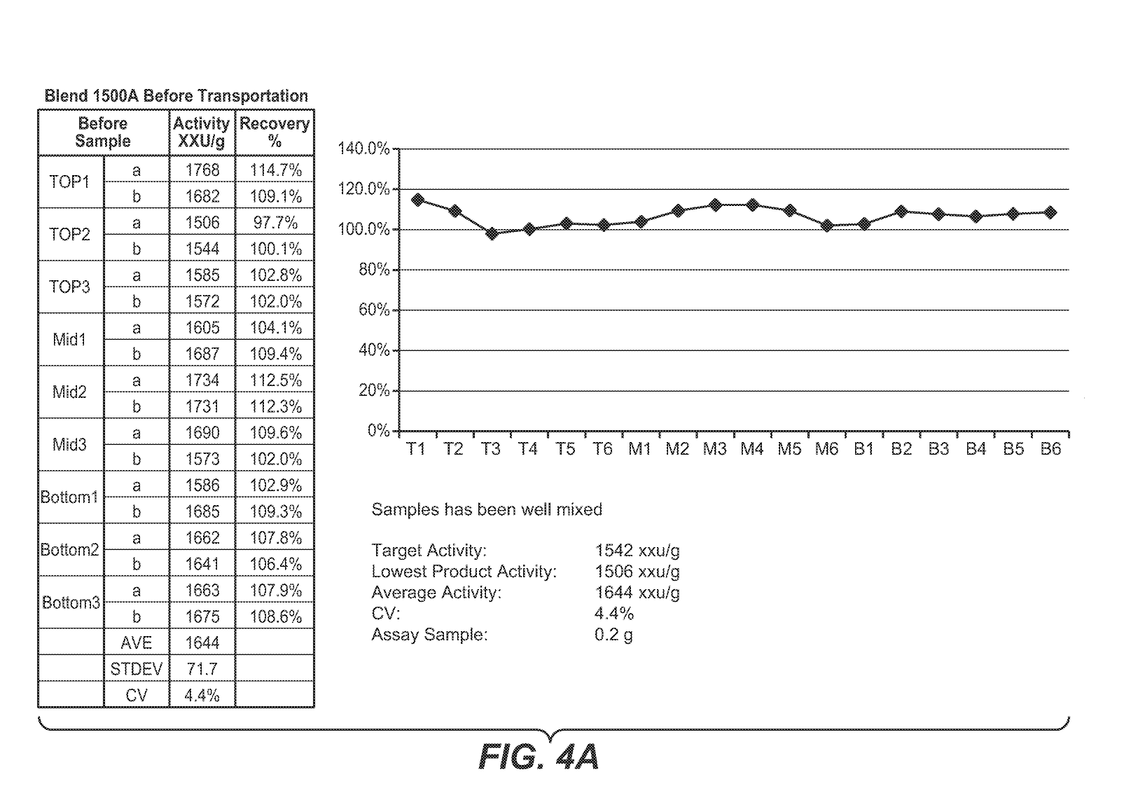

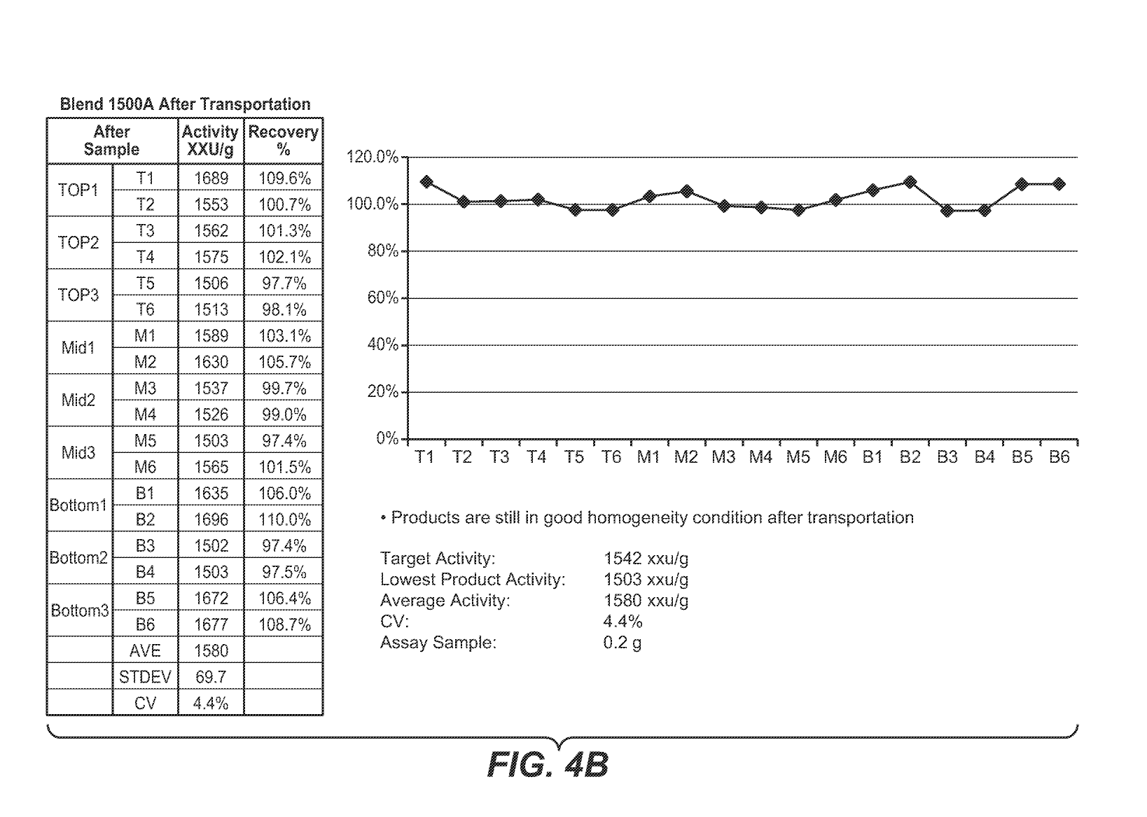

A segregation test was performed to determine whether enzyme granules and dummy granules remain homogeneously blended after mixing and during transportation. A 20 kilogram sample of Purafect 1500A was produced by blending 15 kg of Purafect 2000A with 5 kg of Hanhua −40/+60 mesh sodium sulfate seeds. The Purafect 1500A blend was placed in a 30 liter drum and mixed for 10 minutes. 9 samples were taken from the stream of material as it was poured from the drum into a carton. The carton was placed in the trunk of a car and driven for 150 kilometers over 3 days over normal road conditions involving driving and shaking. Nine samples were taken from locations at the top (T) middle (M) and bottom (B) of the carton. The original nine samples from filling and the final nine samples after transportation were analyzed for enzyme activity, and the results are tabulated and plotted in FIGS. 4A and 4B.

FIGS. 4A and 4B show no difference in the coefficient of variation (CV) across nine samples taken before and after transportation—the CV is 4.4% in both cases. This demonstrates that no appreciable segregation is induced in the enzyme-dummy particle blend by means of the normal vibration and shaking induced by normal driving conditions.

### Example 5

### Flow Properties of Enzyme Granules, Diluent Particles, and Blends

A granule flowability study was conducted to determine how well an enzyme granule—dummy particle blend would flow under conditions simulating flow in a plant blender or metering system. Ten ml volume of particles were loaded into a glass funnel and allowed to flow freely through a standard glass buret with a 2 mm inner diameter. Flow rate was measured as the number of seconds required to empty the 10 ml sample through the buret.

Flowabilty tests were performed on two enzyme granules (Purafast 2000A, Properase 2000A), a dummy particle (Hanhua −40/+60 mesh sodium sulfate) a previously prepared blend of enzyme granules and dummy granules (Purafast 1500A) and a blend prepared on the spot (75% Purafast 2000A+25% dummy particles). Three repeat runs of each sample were performed, and the flowability measurements were averaged.

The results show that the flowability of the enzyme granule—dummy granule blend is equivalent to that of unblended enzyme granules, even though the dummy granule by itself flows more slowly. This suggests that the flowability of a mixture is not a linear combination of the flowabilities of the individual mixture components.

### Example 6

### Moisture Uptake of Enzyme Granules and Blends

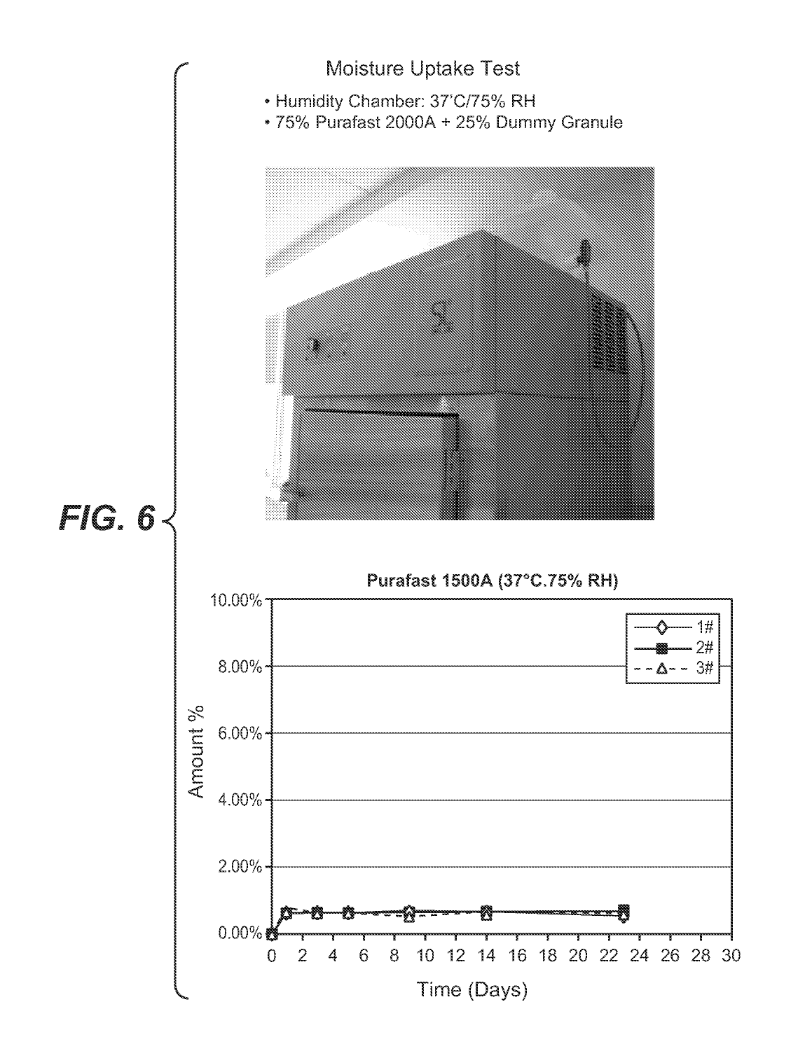

FIG. 6 shows the moisture uptake of a blend of 75% Purafect 2000A enzyme granules with 25% Hanhua −40/+60 mesh sodium sulfate crystals during 23 days storage at 37° C., 75% relative humidity. As can be seen, the blend absorbs less than 1% w/w moisture under these conditions.

### Example 7

### Storage Stability and Moisture Uptake of Enzyme Granule Blends in Detergent

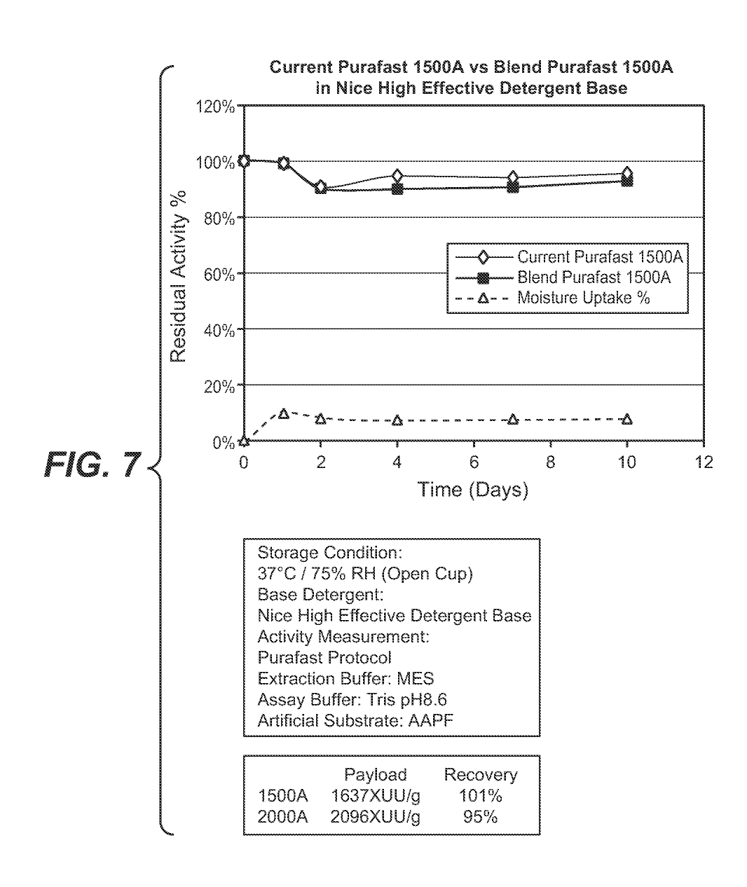

FIG. 7 compares the storage stability of an enzyme granule- dummy particle blend (Purafect 1500A blend, produced as a mixture of 75% Purafast 2000A and 25% Hanhua =40/+60 mesh sodium sulfate crystals) compared with that of an equivalent strength unblended enzyme granule (“Current” Purafect 1500A) after storage in commercial available Nice high effective HDD detergent during 10 days storage at 37° C. and 75% relative humidity. Also shown are concurrent measurements of gravimetric moisture uptake in the detergent. As can be seen, there is no significant difference in enzyme stability of the new enzyme—dummy granule blend vs. the equivalent strength unblended enzyme granule.

### Example 8

### Visual Appearance

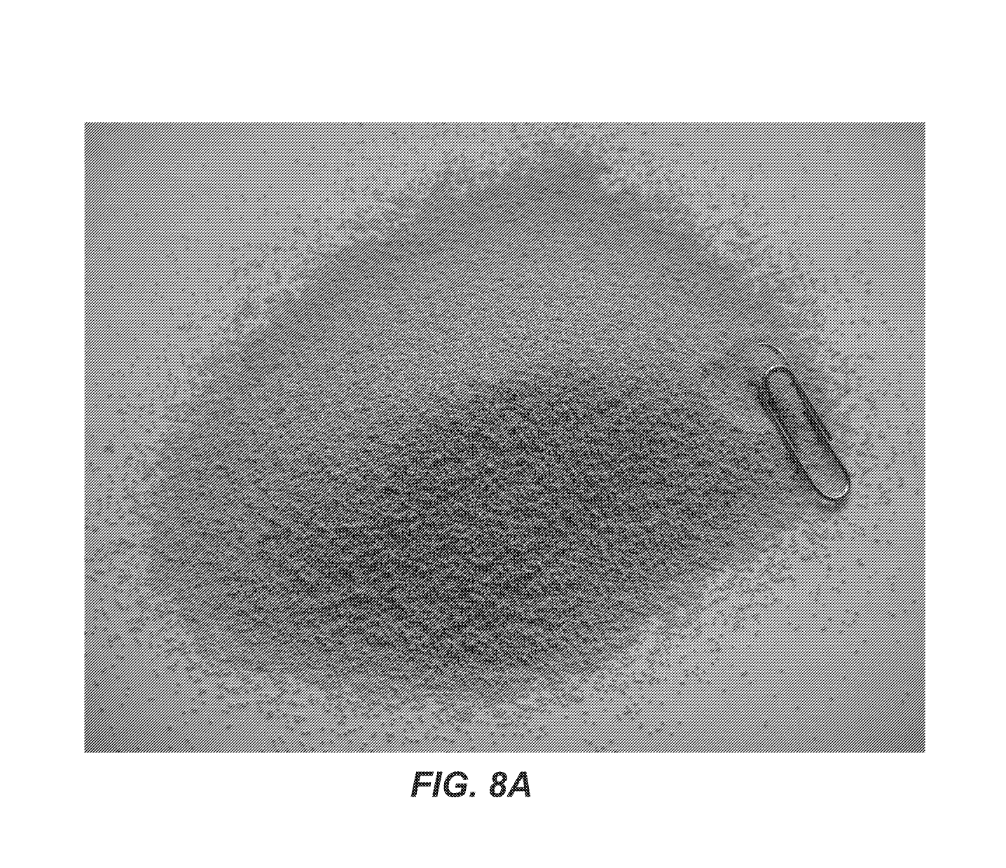

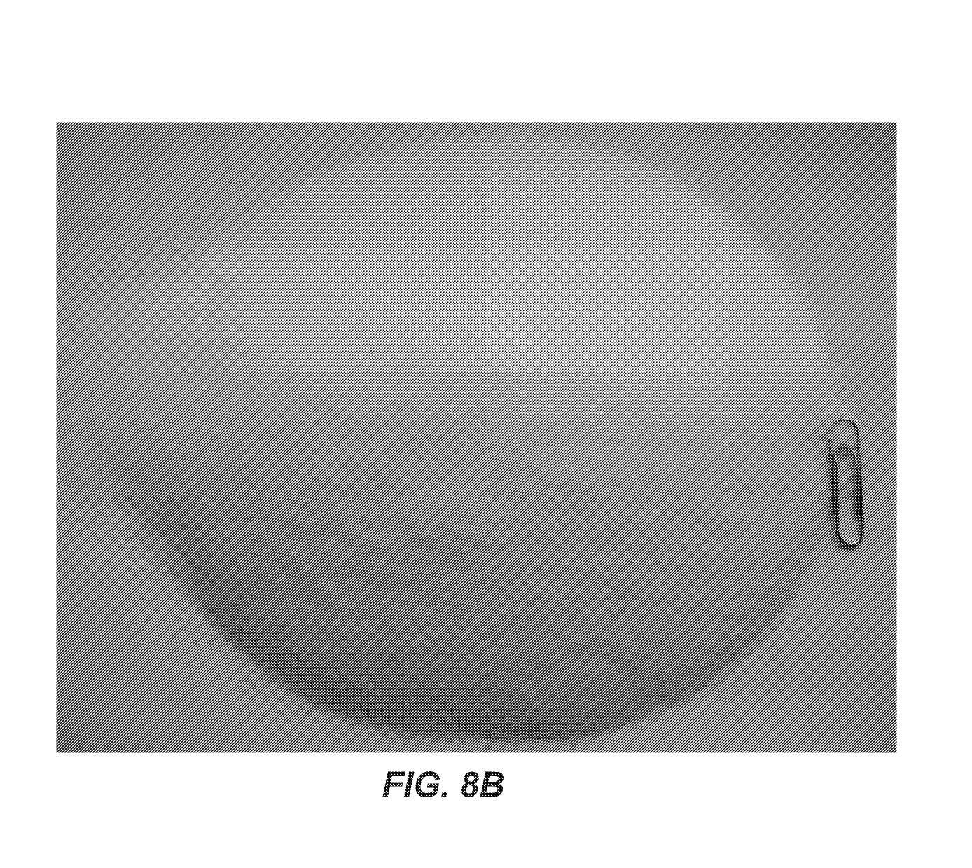

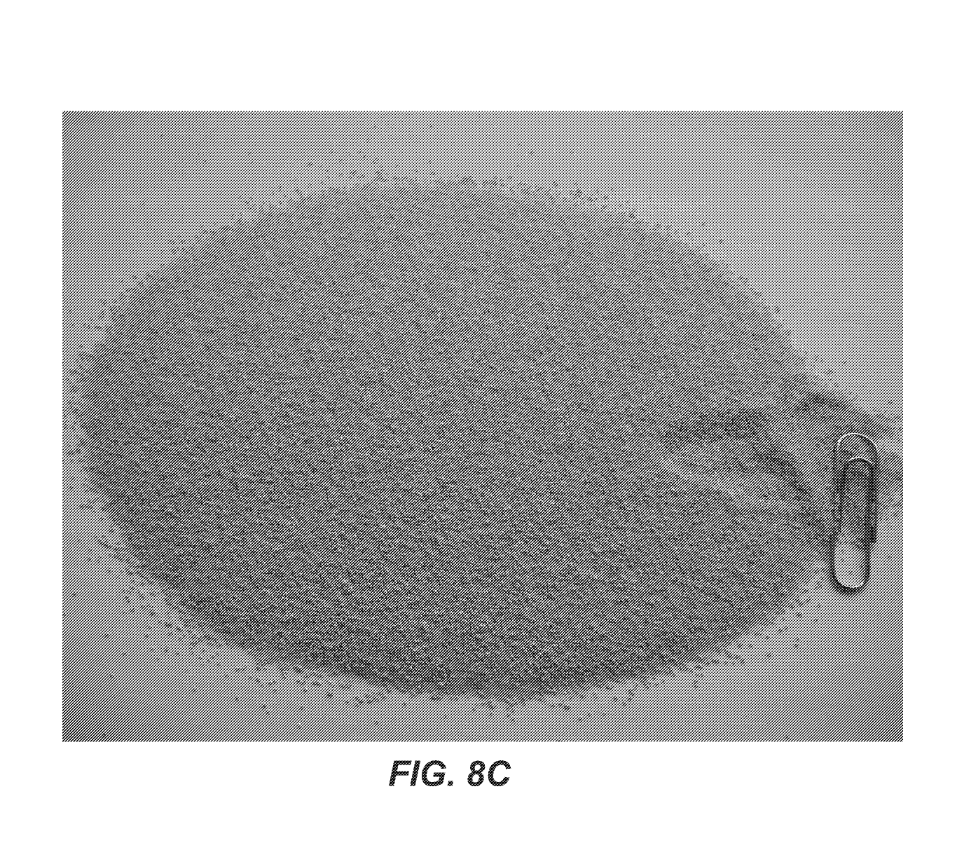

Even though neat samples of Purafect 2000A and Hanhua −40/+60 mesh sodium sulfate crystals (“dummy particles) appear distinct, a blend of 75% Purafect 2000A with 25% Hanhua −40/+60 mesh sodium sulfate crystals appears to be visually homogeneous, as can be seen by photo in FIGS. 8A-8C.

1-7. (canceled)

8. A mixture consisting essentially of;
a collection of small enzyme granules comprising a sodium sulfate core and at least one layer, wherein at least 80% of the small enzyme granules comprise a diameter of about 300-400 microns; and,
a collection of size-matched sodium sulfate dummy particle, wherein at least 80% of the size-matched sodium sulfate dummy particle comprises a diameter of about 300-400 microns,
wherein the median size of the small enzyme granules and the median size of the sodium sulfate dummy particles are size-matched such that they vary by less than 20 microns.

9. The mixture of claim 8 wherein the enzyme is a protease.

10. The mixture of claim 8 wherein the enzyme is a phytase.

11. A method of washing dishes comprising contacting the dishes with the mixture of claim 8.

12. A method of washing clothes comprising contacting the clothes with the mixture of claim 8.

14. A method of feeding animals comprising providing an animal feed to an animal in need of such feed, wherein the feed comprises the mixture according to claim 8.
